(19) United States
(12) Patent Application Publication
(10) Pub. No.: US 2004/0223925 A1
(43) Pub. Date: Nov. 11, 2004

(54) Water-in-oil photoprotective emulsions comprising polyolefinic surfactants and 4,4-diarylbutadiene UV-A sunscreens

(75) Inventor: Florence L'Alloret, Paris (FR)

Correspondence Address: BURNS DOANE SWECKER & MATHIS L L P, POST OFFICE BOX 1404, ALEXANDRIA, VA, 22313-1404, US

(73) Assignee: L'OREAL, PARIS (FR)

(21) Appl. No.: 10/823,735

(22) Filed: Apr. 14, 2004

Related U.S. Application Data
(60) Provisional application No. 60/468,114, filed on May 6, 2003.

(30) Foreign Application Priority Data
Apr. 14, 2003 (FR) 03/04649

(51) Int. Cl. A61K 7/42

(52) U.S. Cl. 424/59

(57) ABSTRACT

Stable UV-photoprotective water-in-oil emulsions, well suited for the photoprotection of the skin, lips and/or hair against the damaging effects of UV-radiation, contain at least one polymeric surfactant comprising at least one polar part and at least one polyolefinic apolar part, and also containing at least one organic 4,4-diarylbutadiene UV-A-screening agent.

### CROSS-REFERENCE TO PRIORITY/PROVISIONAL APPLICATIONS

This application claims priority under 35 U.S.C. § 119 of FR 03/04649, filed Apr. 14, 2003, and of provisional application Ser. No. 60/468,114, filed May 6, 2003, both hereby expressly incorporated by reference and both assigned to the assignee hereof. This application is also a continuation of said '114 provisional.

### BACKGROUND OF THE INVENTION

1. Technical Field of the Invention

The present invention relates to water-in-oil photoprotective emulsions comprising at least one polymeric surfactant which comprises at least one polar part or moiety and at least one polyolefinic apolar part or moiety and which also comprise at least one organic UV-A-screening agent of the 4,4-diarylbutadiene type.

2. Description of Background and/or Related and/or Prior Art

It is well known that light radiation having wavelengths of between 280 and 400 nm allows tanning of the human epidermis, and that rays having wavelengths of between 280 and 320 nm, known by the name UV-B, cause erythemas and skin burns which can hamper the development of the natural tan; this UV-B radiation must therefore be screened out.

It is also known that UV-A rays having wavelengths of between 320 and 400 nm, which cause tanning of the skin, are capable of inducing its impairment, radiation. UV-A rays cause in particular a loss of elasticity of the skin and the appearance of wrinkles which lead to premature aging. They promote the onset of the erythematous reaction or amplify this reaction in some subjects and may even be responsible for phototoxic or photoallergic reactions. It is therefore desirable to also screen out UV-A radiation.

The UV-A and UV-B rays must therefore be screened out and protective cosmetic compositions for the human epidermis containing UV-A- and UV-B-screening agents currently exist.

Among the galenic forms available to the formulator, water-in-oil emulsions are particularly suitable because they can form a lipid film at the surface of the skin which is thus protected, or can bring about good stability of the UV-screening agents at the skin surface.

Polymeric surfactants comprising at least a polar part and at least a polyolefinic apolar part make it possible to manufacture water-in-oil emulsions which are stable during storage and are suitable for cosmetic, dermatological and/or pharmaceutical applications. In particular, they make it possible to manufacture water-in-oil emulsions which are very rich in aqueous phase (more than 80%) and which are stable, or water-in-oil emulsions of low viscosity and which are stable. These surfactants thus have advantages compared with the emulsifying agents commonly used for stabilizing water-in-oil emulsions, in particular alkyl polyglycerol, alkyl POE, alkyl sorbitan, metal salts of fatty acids and silicone surfactants. Indeed, for these emulsifiers, the content of aqueous phase is in general less than 80%, the surfactant concentrations are high in order to bring about good stability of the emulsions and the fatty phase comprises a major proportion of silicone oils when silicone surfactants are used. Moreover, the water-in-oil emulsions stabilized with a surfactant derived from polyolefins possess particularly good cosmetic properties, with a light and refreshing feel, without being silicone-based.

Among the available organic UV-A-screening agents, a family of compounds which are particularly effective in the UV-A region is 1,4-benzene[di(3-methylidene-10-camphorsulphonic)]acid and its different salts, which is described in particular in FR-A-2,528,420 and FR-A-2,639,347; they are indeed capable of absorbing ultraviolet rays having wavelengths of between 280 and 400 nm, with absorption maxima of between 320 and 400 nm, in particular in the region of 345 nm.

However, the introduction of this UV-A-screening agent into these emulsions can limit their stability, in particular their resistance to temperature cycles, or can completely prevent the formation of an emulsion during manufacture.

It thus appears necessary to provide water-in-oil emulsions based on polymeric surfactants derived from stable polyolefins which may contain organic screening agents active in the UV-A region and of comparable efficacy to that of 1,4-benzene[di(3-methylidene-10-camphorsulphonic)]acid and its different salts without the disadvantages listed above.

### SUMMARY OF THE INVENTION

It has now surprisingly and unexpectedly been determined that water-in-oil emulsions comprising at least one polymeric surfactant derived from a polyolefin and at least one UV-A-screening agent of the 4,4-diarylbutadiene type ameliorate or avoid those disadvantages and drawbacks indicated above.

This discovery forms the basis of the present invention.

Thus, the present invention features water-in-oil photoprotective emulsions comprising at least one polymeric surfactant which comprises at least one polar part or moiety and at least one polyolefinic apolar part or moiety, and which also comprise at least one organic 4,4-diarylbutadiene UV-A-screening agent.

Other characteristics, aspects and advantages of the invention will be seen from the detailed description which follows.

The expression “stable emulsion” is understood to mean, for the purposes of the present invention, any emulsion whose macroscopic and microscopic appearance is not modified after 1 month at room temperature.

### DETAILED DESCRIPTION OF BEST MODE AND SPECIFIC/PREFERRED EMBODIMENTS OF THE INVENTION

The polymeric surfactants of the invention comprise at least one polar part or moiety and at least one polyolefinic apolar part or moiety. They have in general a block or comb type structure.

The polyolefinic apolar part/moiety may be selected from among polymers and/or copolymers of ethylene, propylene, 1-butene, isobutene, 1-pentene, 2-methyl-1-butene, 3-methyl-1-butene, 1-hexene, 1-heptene, 1-octene, 1-decene, 1-undecene, 1-dodecene, 1-tridecene, 1-tetradecene, 1-pentadecene, 1-hexadecene, 1-heptadecene and 1-octadecene. The polymer chains are hydrogenated or not. They contain at least 40 carbons, and preferably of 60 to 700 carbons.

The polar part/moiety of the oligomers or polymers of the invention may be anionic, cationic, nonionic, zwitterionic or amphoteric. It comprises for example polyalkylene glycols or polyalkyleneimines, or alternatively of carboxylic or dicarboxylic acids, anhydrides thereof or derivatives thereof, and mixtures thereof. The oligomers or polymers with a carboxylic acid polar part/moiety may be obtained for example from the reaction between a polyolefin and at least one carboxylic acid or anhydride selected from the group consisting of maleic acid, maleic anhydride, fumaric acid, itaconic acid, citraconic acid, mesaconic acid and aconitic acid. Preferably, the polar part/moiety comprises succinic acid or anhydride, their ester or amide derivatives, the corresponding salts of alkali metal, alkaline earth metal or organic ions, or alternatively of polyoxyethylene.

The polymeric surfactants of the invention are for example polyisoprene-polyoxyethylene diblock polymers or poly(ethylene-co-propylene)-polyoxyethylene polymers described in the publication by Allgaier, Poppe, Willner, Richter (Macromolecules, 1997, 30, p. 1582-1586).

The polymeric surfactants of the invention are also selected from among the polymers derived from succinic acid or anhydride described in U.S. Pat. Nos. 4,234,435, 4,708,753, 5,129,972, 4,931,110, GB-A-2,156,799 and U.S. Pat. No. 4,919,179. The polyolefin part/moiety may comprise, for example, of polyisobutylene, hydrogenated or not, having a molecular weight ranging from 400 to 5000. In the polyisobutylene with a succinic endgroup thus obtained, the succinic part may be esterified, amidated or in the form of a salt, that is to say that it can be modified with alcohols, amines, alkanolamines or polyols, or alternatively may be in the form of salts of an alkali metal or an alkaline earth metal, or ammonium or alternatively of an organic base such as the salts of diethanolamine and triethanolamine. The polyolefins with an esterified or amidated succinic endgroup are products of the reaction of (a) a polyolefin with a succinic endgroup, and of (b) an amine or an alcohol, to form an amide or an ester. The term “amine” used here comprises all types of amines including alkanolamines. This may include for example primary, secondary or tertiary monoamines, it being possible for these amines to be aliphatic, cycloaliphatic, aromatic, heterocyclic, saturated or unsaturated. Moreover, the alcohols may be mono- or polyalcohols. The monoalcohols comprise the primary, secondary or tertiary aliphatic alcohols, and phenols. The polyalcohols may be for example selected from aliphatic, cycloaliphatic, aromatic and heterocyclic polyalcohols. Polyolefins with a modified (esterified or amidated) succinic endgroup and their method of preparation are described in particular in U.S. Pat. No. 4,708,753.

As polyolefins with a succinic endgroup, there may be mentioned in particular polyisobutylenes with an esterified succinic endgroup and their salts, in particular diethanolamine salts, such as the products marketed under the names Lubrizol 2724, Lubrizol 2722 and Lubrizol 5603 (INCI name: Hydroxyethyldiethonium polyisobutenyl triethylaminosuccinate (and) diethyl ethanolamine) by Lubrizol.

Another example of a polymeric surfactant which can be used in the invention is the product of the reaction of maleic anhydride with polyisobutylene, such as Glissopal SA marketed by BASF.

The polyolefinic surfactants in accordance with the invention make it possible in general to reduce the (water/oil) interfacial tension by at least 10 mN/m at a concentration of 0.01% by weight relative to the total weight of the oily phase. For example, the polyolefin with a succinic endgroup marketed under the name Lubrizol 2724 by Lubrizol, at a concentration of 0.01% by weight relative to the total weight of the oily phase, reduces the interfacial tension by 15 mN/m at the interface of an aqueous phase comprising a 1% aqueous solution of MgSO4, and of an oily phase containing a mixture of oils (isohexadecane/hydrogenated polyisobutene/volatile silicone in a ratio of 8/6/4).

The concentration by mass of surfactant derived from a polyolefin ranges preferably from 0.1% to 10%, and more preferably from 1% to 3% of active material relative to the total weight of the composition. They can be used alone or as a mixture with amphiphilic agents which can stabilize water-in-oil emulsions (surfactants, polymers, particles).

The 4,4-diarylbutadiene compounds in accordance with the invention are preferably selected from among those corresponding to the following formula (I):

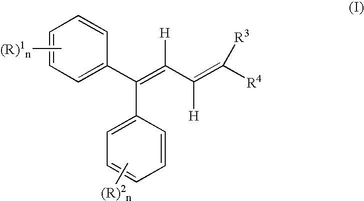

in which the diene system is of the Z,Z; Z,E; E,Z or E,E configuration or mixtures of the said configurations, and wherein:

R¹ and R², which may be identical or different, are each hydrogen, a C₁-C₂₀ alkyl radical, a C₂-C₁₀ alkenyl radical, a C₁-C₁₂ alkoxy radical, a C₃-C₁₀ cycloalkyl radical, a C₃-C₁₀ cycloalkenyl radical, a C₁-C₂₀ alkoxycarbonyl radical, a C₁-C₁₂ monoalkylamino radical, a C₁-C₁₂ dialkylamino radical, an aryl radical, a heteroaryl radical or a water-solubilizing substituent selected from among a carboxylate residue, a sulphonate residue or an ammonium residue;

R³ is a group COOR⁵, COR⁵, CONR⁵R⁶, CN, O═S(—R⁵)═O, O═S(—OR⁵)═0, R⁷O—P—(—OR⁸)═O, a C₁-C₂₀ alkyl radical, a C₂-C₁₀ alkenyl radical, a C₃-C₁₀ cycloalkyl radical, a C₇-C₁₀ bicycloalkyl radical, a C₃-C₁₀ cycloalkenyl radical, a C₇-C₁₀ bicycloalkenyl radical, an optionally substituted C₆-C₁₈ aryl radical, an optionally substituted C₃-C₇ heteroaryl radical;

R⁴ is a group COOR⁶, COR⁶, CONR⁵R⁶, CN, O═S(—R⁶)═O, O═S(—OR⁶)═0, R⁷O—P—(—OR⁸)═O, a C₁-C₂₀ alkyl radical, a C₂-C₁₀ alkenyl radical, a C₃-C₁₀ cycloalkyl radical, a C₇-C₁₀ bicycloalkyl radical, a C₃-C₁₀ cycloalkenyl radical, a C₇-C₁₀ bicycloalkenyl radical, an optionally substituted C₆-C₁₈ aryl radical, an optionally substituted C₃-C₇ heteroaryl radical;

the radicals R⁵ to R⁸, which may be identical or different, are each hydrogen, a C₁-C₂₀ alkyl radical, a C₂-C₁₀ alkenyl radical, a C₃-C₁₀ cycloalkyl radical, a C₇-C₁₀ bicycloalkyl radical, a C₃-C₁₀ bicycloalkenyl radical, a C₇-C₁₀ cycloalkenyl radical, an optionally substituted aryl radical, an optionally substituted heteroaryl radical;

n ranges from 1 to 3; with the proviso that the radicals R³ to R⁸ can together form, with the carbon atoms from which they depend, a C₅-C₆ ring which may be fused.

As C₁-C₂₀ alkyl radicals, there may be mentioned, for example: methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,2,2-trimethylpropyl, 1-ethyl-1-methylpropyl, 1-ethyl-2-methylpropyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, n-nonadecyl or n-eicosyl.

As C₂-C₁₀ alkenyl groups, there may be mentioned, for example: ethenyl, n-propenyl, 1-methylethenyl, n-butenyl, 1-methylpropenyl, 2-methylpropenyl, 1,1-dimethylethenyl, n-pentenyl, 1-methylbutenyl, 2-methylbutenyl, 3-methylbutenyl, 2,2-dimethylpropenyl, 1-ethylpropenyl, n-hexenyl, 1,1-dimethylpropenyl, 1,2-dimethylpropenyl, 1-methylpentenyl, 2-methylpentenyl, 3-methylpentenyl, 4-methylpentenyl, 1,1-dimethylbutenyl, 1,2-dimethylbutenyl, 1,3-dimethylbutenyl, 2,2-dimethylbutenyl, 2,3-dimethylbutenyl, 3,3-dimethylbutenyl, 1-ethylbutenyl, 2-ethylbutenyl, 1,1,2-trimethylpropenyl, 1,2,2-trimethylpropenyl, 1-ethyl-1-methylpropenyl, 1-ethyl-2-methylpropenyl, n-heptenyl, n-octenyl, n-nonenyl, n-decenyl.

As C₁-C₁₂ alkoxy radicals, there may be mentioned: methoxy, n-propxy, 1-methylpropoxy, 1-methylethoxy, n-pentoxy, 3-methylbutoxy, 2,2-dimethylpropoxy, 1-methyl-1-ethylpropoxy, octoxy, ethoxy, n-propoxy, n-butoxy, 2-methylpropoxy, 1,1-dimethylpropoxy, hexoxy, heptoxy, 2-ethylhexoxy.

As C₁-C₂₀ alkoxycarbonyl radicals, there may be mentioned esters of C₁-C₂₀ alcohols.

As C₁-C₁₂ monoalkylamino or dialkylamino radicals, there may be mentioned those in which the alkyl radical(s) is (are) selected from among methyl, n-propyl, 2-methylpropyl, 1,1-dimethylethyl, hexyl, heptyl, 2-ethylhexyl, isopropyl, 1-methylpropyl, n-pentyl, 3-methylbutyl, 2,2-dimethylpropyl, 1-methyl-1-ethylpropyl, octyl.

As C₃-C₁₀ cycloalkyl radicals, there may be mentioned, for example: cyclopropyl, cyclobutyl, cyclopentyl, cyclohexyl, cycloheptyl, 1-methylcyclopropyl, 1-ethylcyclopropyl, 1-propylcyclopropyl, 1-butylcyclopropyl, 1-pentylcyclopropyl, 1-methyl-1-butylcyclopropyl, 1,2-dimethylcyclopropyl, 1-methyl-2-ethylcyclopropyl, cyclooctyl, cyclononyl or cyclodecyl.

As C₃-C₁₀ cycloalkenyl radicals having one or more double bonds, there may be mentioned: cyclobutenyl, cyclopentenyl, cyclopentadienyl, cyclohexenyl, 1,3-cyclohexadienyl, 1,4-cyclohexadienyl, cycloheptenyl, cycloheptatrienyl, cyclooctenyl, 1,5-cyclooctadienyl, cyclooctetraenyl, cyclononenyl or cyclodecenyl.

The cycloalkyl or cycloalkenyl radicals may comprise one or more substituents (preferably from 1 to 3) selected for example from among halogens such as chlorine, fluorine or bromine; cyano; nitro; amino; C₁-C₄ alkylamino; C₁-C₄ dialkylamino; C₁-C₄ alkyl; C₁-C₄ alkoxy; hydroxyl; they may also comprise from 1 to 3 heteroatoms such as sulphur, oxygen or nitrogen whose free valencies may be saturated with a hydrogen or a C₁-C₄ alkyl radical.

The bicycloalkyl or bicycloalkenyl groups are selected, for example, from among bicyclic terpenes such as pinane, bornane, pinene or camphor or adamantane derivatives.

The aryl groups are preferably selected from phenyl or naphthyl rings, which may comprise one or more substituents (preferably from 1 to 3) selected for example from halogen such as chlorine, fluorine or bromine; cyano; nitro; amino; C₁-C₄ alkylamino; C₁-C₄ dialkylamino; C₁-C₄ alkyl; C₁-C₄ alkoxy; hydroxyl. Phenyl, methoxyphenyl, naphthyl and thienyl are more particularly preferred.

The heteroaryl groups comprise in general one or more heteroatoms selected from among sulphur, oxygen or nitrogen.

The water-solubilizing groups are, for example, carboxyl and sulphoxy residues, and more particular their salts with physiologically acceptable cations such as alkali metal salts or trialkylammonium salts such as tri(hydroxyalkyl)ammonium or 2-methylpropan-1-ol-2-ammonium salts. There may also be mentioned ammonium groups such as alkylammoniums and their salified forms with physiologically acceptable anions.

The compounds of formula (I) are known per se and their structures and their syntheses are described in DE-1-9,755,649, EP-916,335, EP-1,133,980 and EP-1,133,981.

By way of example of a compound of formula (I), the following are representative:

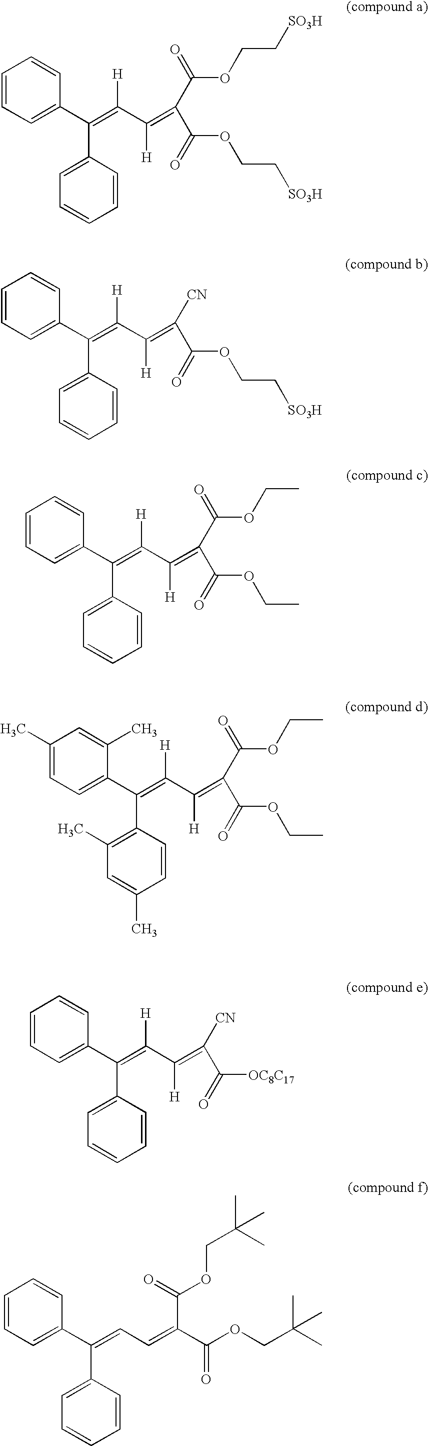

The preferred compounds of formula (I) are those for which n=1 or 2;

R¹ and R², which may be identical or different, are each hydrogen, a C₁-C₂₀ alkyl radical, a C₁-C₁₂ alkoxy radical, a C₁-C₁₂ monoalkylamino radical, a C₁-C₁₂ dialkylamino radical, a water-solubilizing substituent selected from among a carboxylate group, a sulphonate group or an ammonium residue;

R³ is a group COOR⁵, COR⁵, CONR⁵R⁶, a C₁-C₂₀ alkyl radical, a C₃-C₁₀ cycloalkyl radical, a C₃-C₁₀ cycloalkenyl radical, a C₇-C₁₀ bicycloalkyl radical, optionally substituted phenyl, naphthyl or thienyl;

R⁴ is a group COOR⁶, COR⁶, CONR⁵R⁶, a C₁-C₂₀ alkyl radical, a C₃-C₆ cycloalkyl radical, a C₃-C₁₀ cycloalkenyl radical, a C₇-C₁₀ bicycloalkyl radical, optionally substituted phenyl, naphthyl or thienyl;

the radicals R⁵ and R⁶, which may be identical or different, are each hydrogen, a C₁-C₁₂ alkyl radical, a C₃-C₁₀ cycloalkyl radical, a C₃-C₁₀ cycloalkenyl radical, a C₇-C₁₀ bicycloalkyl radical, a C₃-C₁₀ bicycloalkenyl radical, optionally substituted phenyl or naphthyl.

Among these compounds, there are more particularly preferred those in which: R¹ and R², which may be identical or different, are each hydrogen, a C₁-C₂₀ alkyl radical, a C₁-C₂₀ alkoxy radical, a water-solubilizing substituent selected from among a carboxylate group, a sulphonate group or an ammonium residue;

R³ is a group COOR⁵, COR⁵, CONR⁵R⁶;

R⁴ is a group COOR⁶, COR⁶, CONR⁵R⁶; and

the radicals R⁵ and R⁶, which may be identical or different, are each hydrogen, a C₁-C₁₂ alkyl radical, a C₃-C₆ cycloalkyl radical, a C₃-C₁₀ cycloalkenyl radical, a C₇-C₁₀ bicycloalkyl radical, a C₃-C₁₀ bicycloalkenyl radical, optionally substituted phenyl or naphthyl.

According to a particularly preferred embodiment, the compounds of formula (I) are selected from among those of the following formula (I′):

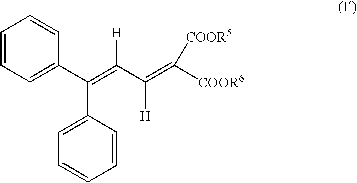

wherein the radicals R⁵ and R⁶, which may be identical or different, are each hydrogen, a C₁-C₂₀ alkyl radical, a C₃-C₆ cycloalkyl radical, a C₃-C₁₀ cycloalkenyl radical.

Among these compounds of formula (I′), there may be mentioned more particularly 1,1-dicarboxy(2,2′-dimethylpropyl)-4,4-diphenylbutadiene having the structure:

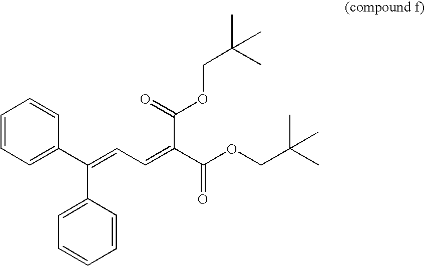

Another 4,4-diarylbutadiene family which may be formulated into the emulsions according to the invention are those corresponding to the following formula (II):

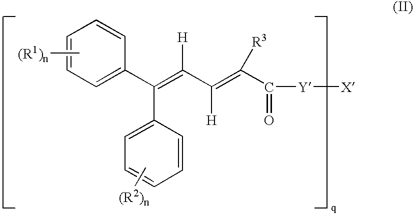

in which the diene system is of the Z,Z; Z,E; E,Z or E,E configuration or mixtures of the said configurations and wherein:

R¹, R², R³ and n have the same meanings indicated in the preceding formula (I);

Y′ is a group —O— or —NR⁹—;

R⁹ is hydrogen, a linear or branched C₁-C₂₀ alkyl radical, a C₂-C₁₀ alkenyl radical, a C₃-C₁₀ cycloalkyl radical, a C₇-C₁₀ bicycloalkyl radical, a C₃-C₁₀ cycloalkenyl radical, a C₇-C₁₀ bicycloalkenyl radical, an aryl radical, a heteroaryl radical;

X′ is a residue of a linear or branched, aliphatic or cycloaliphatic polyol C₂-C₂₀ comprising from 2 to 10 hydroxyl groups and having the valency q; with the proviso that the carbon chain of the said residue to be interrupted by one or more sulphur or oxygen atoms, one or more imine groups, one or more C₁-C₄ alkylimino groups;

q ranges from 2 to 10; and

X′ is a polyol C₂-C₂₀ residue containing from 2 to 10 hydroxyl groups, and in particular:

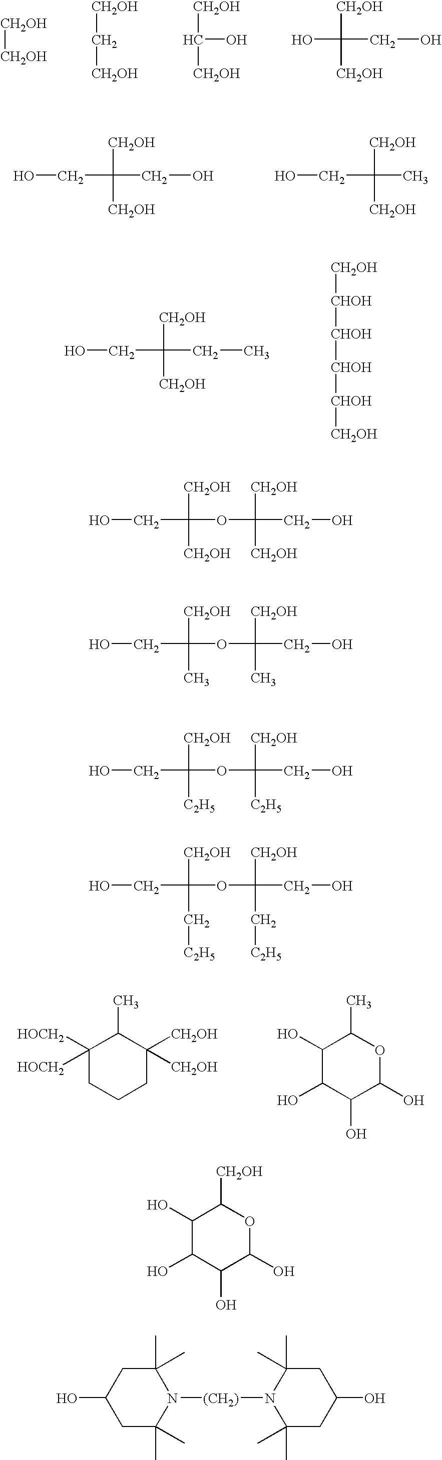

The more preferred compounds of formula (II) are those for which: R¹ and R², which may be identical or different, are each hydrogen, a C₁-C₁₂ alkyl radical, a C₁-C₈ alkoxy radical, a water-solubilizing substituent selected from among a carboxylate group, a sulphonate group or an ammonium residue;

R³ is a group COOR⁵, CONR⁵R⁶, CN, a C₃-C₁₀ cycloalkyl radical, a C₇-C₁₀ bicycloalkyl radical;

R⁵ and R⁶, which may be identical or different, are each a linear or branched C₁-C₂₀ alkyl radical, a C₃-C₁₀ cycloalkyl radical, a C₇-C₁₀ bicycloalkyl radical, optionally substituted naphthyl or phenyl;

X′ is a polyol C₂-C₂₀ residue comprising from 2 to 6 hydroxyl groups and more particularly from 2 to 4.

The even more preferred compounds of formula (II) are those for which: X′ is an ethanol or pentaerythrol residue.

The even more particularly preferred compounds of formula (II) are selected from among:

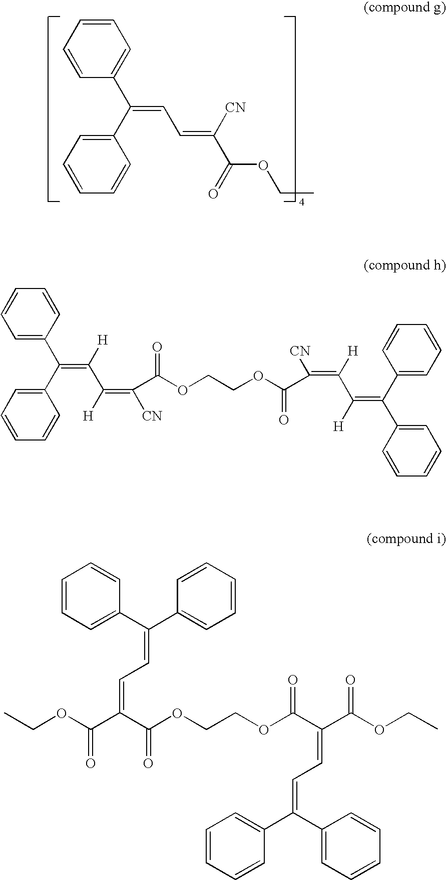

The compounds of formula (II) as defined above are known per se and their structures and their syntheses are described in EP-A-1-008,586.

The 4,4-diarylbutadiene compounds are preferably present in the composition in proportions ranging from 0.1% to 20% by weight, more preferably from 1% to 10% by weight relative to the total weight of the composition.

The nature of the fatty phase entering into the composition of the emulsions according to the invention is not critical and it may thus comprise all the compounds which are already known in general as being suitable for the manufacture of water-in-oil type emulsions. In particular, these compounds may be selected, alone or as mixtures, from among various fatty substances, oils of plant, animal or mineral origin, natural or synthetic waxes, and the like.

Among the oils which may comprise the composition of the fatty phase, there may be mentioned in particular:

mineral oils such as paraffin oil and liquid paraffin,

oils of animal origin, such as perhydrosqualene,

oils of plant origin, such as sweet almond oil, avocado oil, castor oil, olive oil, jojoba oil, sesame oil, groundnut oil, grape seed oil, rapeseed oil, copra oil, hazelnut oil, shea butter, palm oil, apricot kernel oil, calophyllum oil, rice bran oil, maize germ oil, wheat germ oil, soya bean oil, sunflower oil, evening primrose oil, safflower oil, passion flower oil and rye oil,

synthetic oils, such as purcellin oil, esters, such as for example butyl myristate, isopropyl myristate, cetyl myristate, isopropyl palmitate, isopropyl adipate, ethylhexyl adipate, butyl stearate, hexadecyl stearate, isopropyl stearate, octyl stearate, isocetyl stearate, decyl oleate, hexyl laurate, propylene glycol dicaprylate and esters derived from lanolic acid such as isopropyl lanolate, isocetyl lanolate, isoparaffins and poly-α-olefins.

As other oils which can be formulated into the emulsions according to the invention, there may also be mentioned C12-C15 fatty alcohol benzoates (Finsolv TN from FINETEX), ethers, lipophilic amino acid derivatives such as isopropyl N-lauroylsarcosinate (Eldew SL-205 from Ajinomoto), fatty alcohols such as lauryl, cetyl, myristyl, stearyl, palmityl or oleyl alcohol and 2-octyldodecanol, acetylglycerides, octanoates and decanoates of alcohols and polyalcohols such as those of glycol and of glycerol, ricinoleates of alcohols and polyalcohols such as those of cetyl, fatty acid triglycerides such as caprylic/capric triglycerides, C10-C18 saturated fatty acid triglycerides, fluorinated and perfluorinated oils, lanolin, hydrogenated lanolin, acetylated lanolin and finally volatile or nonvolatile silicone oils.

Of course, the fatty phase may also contain one or more conventional lipophilic cosmetic adjuvants, such as for example waxes, lipophilic gelling agents, surfactants, organic or inorganic particles, and in particular those which are already customarily used in the manufacture and production of anti-sun cosmetic compositions.

Conventionally, the dispersive aqueous phase has a content of between 40% and 95% by weight. It may comprise water, or a mixture of water and polyhydric alcohol(s) such as for example glycerol, propylene glycol, butylene glycol and sorbitol, or alternatively a mixture of water and water-soluble lower alcohol(s) such as ethanol, isopropanol or butanol (aqueous-alcoholic solution).

The emulsions in accordance with the invention may further comprise other additional organic or inorganic UV-screening agents which are active in UV-A and/or UV-B, which are water-soluble or fat-soluble or alternatively insoluble in the commonly-used cosmetic solvents.

The additional organic screening agents are selected in particular from among anthranilates; cinnamic derivatives; dibenzoylmethane derivatives; salicylic derivatives, camphor derivatives; triazine derivatives such as those described in U.S. Pat. No. 4,367,390, EP-863,145, EP-517,104, EP-570,838, EP-796,851, EP-775,698, EP-878,469, EP-933,376, EP-507,691, EP-507,692, EP-790,243 and EP-944,624; benzophenone derivatives; β,β-diphenyl acrylate derivatives; benzotriazole derivatives; benzalmalonate derivatives; benzimidazole derivatives; imidazolines; bis-benzoazolyl derivatives as described in EP-669,323 and U.S. Pat. No. 2,463,264; p-aminobenzoic acid (PABA) derivatives; methylenebis(hydroxyphenylbenzotriazole) derivatives as described in U.S. Pat. Nos. 5,237,071, 5,166,355, GB-2,303,549, DE-197-26-184 and EP-893,119; benzoxazole derivatives as described in EP-0,832,642, EP-1,027,883, EP-1,300,137 and DE-101-62-844; screening polymers and screening silicones such as those described in particular in WO 93/04665; dimers derived from α-alkylstyrene such as those described in DE-198-55-649 and mixtures thereof.

As examples of organic screening agents which are active in UV-A and/or UV-B, there may be mentioned those designated below under their INCI name:

Para-Aminobenzoic Acid Derivatives:

PABA,

Ethyl PABA,

Ethyl Dihydroxypropyl PABA,

Ethylhexyl Dimethyl PABA sold in particular under the name “ESCALOL 507” by ISP,

Glyceryl PABA,

PEG-25 PABA sold under the name “UVINUL P25” by BASF,

Salicylic Derivatives:

Homosalate sold under the name “Eusolex HMS” by Rona/EM Industries,

Ethylhexyl Salicylate sold under the name “NEO HELIOPAN OS” by Haarmann and REIMER,

Dipropyleneglycol Salicylate sold under the name “DIPSAL” by SCHER,

TEA Salicylate, sold under the name “NEO HELIOPAN TS” by Haarmann and REIMER,

Dibenzoylmethane Derivatives:

Butyl Methoxydibenzoylmethane sold in particular under the trademark “PARSOL 1789” by HOFFMANN LA ROCHE,

Isopropyl Dibenzoylmethane,

Cinnamic Derivatives:

Ethylhexyl Methoxycinnamate sold in particular under the trademark “PARSOL MCX” by HOFFMANN LA ROCHE,

Isopropyl Methoxy cinnamate,

Isoamyl Methoxy cinnamate sold under the trademark “NEO HELIOPAN E 1000” by HAARMANN and REIMER,

Cinoxate,

DEA Methoxycinnamate,

Diisopropyl Methylcinnamate,

Glyceryl Ethylhexanoate Dimethoxycinnamate,

β,β-Diphenyl acrylate derivatives:

Octocrylene sold in particular under the trademark “UVINUL N539” by BASF,

Etocrylene, sold in particular under the trademark “UVINUL N35” by BASF,

Benzophenone Derivatives:

Benzophenone-1 sold under the trademark “UVINUL 400” by BASF,

Benzophenone-2 sold under the trademark “UVINUL D50” by BASF,

Benzophenone-3 or Oxybenzone, sold under the trademark “UVINUL M40” by BASF,

Benzophenone-4 sold under the trademark “UVINUL MS40” by BASF,

Benzophenone-5,

Benzophenone-6 sold under the trademark “Helisorb 11” by Norquay,

Benzophenone-8 sold under the trademark “Spectra-Sorb UV-24” by American

Cyanamid,

Benzophenone-9 sold under the trademark “UVINUL DS-49” by BASF,

Benzophenone-12,

n-Hexyl 2-(4-diethylamino-2-hydroxybenzoyl)benzoate,

Benzylidenecamphor Derivatives:

3-Benzylidene camphor manufactured under the name “MEXORYL SD” by CHIMEX,

4-Methylbenzylidene camphor sold under the name “EUSOLEX 6300” by MERCK,

Benzylidene Camphor Sulphonic Acid manufactured under the name “MEXORYL SL” by CHIMEX,

Camphor Benzalkonium Methosulphate manufactured under the name “MEXORYL SO” by CHIMEX,

Terephthalylidene Dicamphor Sulphonic Acid manufactured under the name “MESORYL SX” by CHIMEX,

Polyacrylamidomethyl Benzylidene Camphor manufactured under the name “MESORYL SW” by CHIMEX,

Benzimidazole Derivatives:

Phenylbenzimidazole Sulphonic Acid sold in particular under the trademark “EUSOLEX 232” by MERCK,

Disodium Phenyl Dibenzimidazole Tetra-sulphonate sold under the trademark “NEO HELIOPAN AP” by Haarmann and REIMER,

Triazine Derivatives:

Anisotriazine sold under the trademark “TINOSORB S” by CIBA SPECIALTY CHEMICALS,

Ethylhexyl triazone sold in particular under the trademark “UVINUL T150” by BASF,

Diethylhexyl Butamido Triazone sold under the trademark “UVASORB HEB” by SIGMA 3V,

2,4,6-Tris(diisobutyl 4′-aminobenzalmalonate)-s-triazine,

Benzotriazole Derivatives:

Drometrizole Trisiloxane sold under the name “Silatrizole” by RHODIA CHIMIE,

Methylene bis-Benzotriazolyl Tetramethylbutylphenol, sold in solid form under the trademark “MIXXIM BB/100” by FAIRMOUNT CHEMICAL or in micronized form in aqueous dispersion under the trademark “TINOSORB M” by CIBA SPECIALTY CHEMICALS,

Anthranilic Derivatives:

Menthyl anthranilate sold under the trademark “NEO HELIOPAN MA” by Haarmann and REIMER,

Imidazoline Derivatives:

Ethylhexyl Dimethoxybenzylidene Dioxoimidazoline Propionate,

Benzalmalonate Derivatives:

Polyorganosiloxane with benzalmalonate functional groups such as polysilicone-15 sold under the trademark “PARSOL SLX” by HOFFMANN LAROCHE

Benzoxazole Derivatives:

2,4-Bis[5-1-(dimethylpropyl)benzoxazol-2-yl-(4-phenyl)imino]-6-(2-ethylhexyl)imino-1,3,5-triazine sold under the name Uvasorb K2A by Sigma 3V; and mixtures thereof.

The organic screening agents which are more particularly preferred are selected from the following compounds:

Ethylhexyl Salicylate,

Ethylhexyl Methoxycinnamate,

Octocrylene,

Butyl Methoxydibenzoylmethane,

Phenylbenzimidazole Sulphonic Acid,

Benzophenone-3,

Benzophenone-4,

Benzophenone-5,

n-Hexyl 2-(4-diethylamino-2-hydroxybenzoyl)benzoate,

4-Methylbenzylidene camphor,

Terephthalylidene Dicamphor Sulphonic Acid,

Disodium Phenyl Dibenzimidazole Tetra-sulphonate,

2,4,6-Tris(diisobutyl 4′-aminobenzalmalonate)-s-triazine,

Anisotriazine,

Ethylhexyl triazone,

Diethylhexyl Butamido Triazone,

Methylene bis-Benzotriazolyl Tetramethylbutylphenol,

Drometrizole Trisiloxane,

Polysilicone 15,

2,4-Bis-[5-1-(dimethylpropyl)benzoxazol-2-yl-(4-phenyl)imino]-6-(2-ethylhexyl)imino-1,3,5-triazine

and mixtures thereof.

The additional inorganic screening agents are selected from among pigments or nanopigments (average size of the primary particles: generally between 5 nm and 100 nm, preferably between 10 nm and 50 nm) of metal oxides, coated or uncoated, such as for example nanopigments of titanium oxide (amorphous or crystallized in rutile and/or anatase form), iron oxide, zinc oxide, zirconium oxide or cerium oxide which are all UV photoprotective agents well known per se. Conventional coating agents are moreover alumina and/or aluminium stearate. Such nanopigments of metal oxides, coated or uncoated, are described in particular in EP-518,772 and EP-518,773.

The additional screening agents according to the invention are generally present in the compositions according to the invention in an amount ranging from 0.1% to 30% by weight, and preferably from 0.5% to 15% by weight, relative to the total weight of the composition.

The compositions according to the invention may also contain agents for artificially bronzing and/or tanning the skin (self-tanning agents).

The self-tanning agents are generally selected from mono- or polycarbonylated compounds such as for example isatin, alloxan, ninhydrin, glyceraldehyde, mesotartaric aldehyde, glutaraldehyde, erythrulose, derivatives of 4,5-pyrazolindiones as described in FR-2,466,492 and WO 97/35842, dihydroxyacetone (DHA), 4,4-dihydroxypyrazolin-5-one derivatives as described in EP-903,342. DHA will preferably be used.

DHA may be used in free form and/or encapsulated for example into lipid vesicles such as liposomes, which are described in particular in WO 97/25970.

The mono- or polycarbonylated self-tanning agents are generally present in the compositions according to the invention in proportions ranging from 0.1% to 10% by weight relative to the total weight of the composition, and preferably from 0.2% to 8% by weight relative to the total weight of the composition.

The compositions in accordance with the present invention may additionally comprise conventional cosmetic adjuvants, selected in particular from among organic solvents, ionic or nonionic thickeners, demulcents, humectants, opacifying agents, stabilizers, emollients, silicones, insect repellents, perfumes, preservatives, surfactants, fillers, active agents, pigments, polymers, propellants, alkanizing or acidifying agents or any other ingredient customarily used in the cosmetic and/or dermatological field.

Of course, those skilled in this art will be careful to select the possible additional compound(s) cited above and/or their quantities such that the advantageous properties intrinsically attached to the compositions in accordance with the invention are not, or not substantially, impaired by the addition(s) envisaged.

Among the organic solvents lower alcohols and polyols may be mentioned.

Among the thickeners, there may be mentioned crosslinked acrylic polymers such as the Carbomers provided by Noveon, acrylate/C10⁻³⁰ alkyl acrylate crosslinked polymers of the Pemulen type provided by Noveon or polyacrylate-3 sold under the name Viscophobe DB 1000 by Amerchol); polymers derived from acrylamido-2-methylpropanesulphonic acid (Hostacerin AMPS provided by Clariant, Sepigel 305 provided by Seppic), synthetic neutral polymers such as poly-N-vinylpyrrolidone, polysaccharides such as guar and xanthan gums, and modified or unmodified cellulose derivatives such as hydroxypropylated guar gum, methylhydroxyethylcellulose and hydroxypropylmethylcellulose.

The compositions according to the invention find application in a large number of treatments, in particular cosmetic treatments, of the skin, the lips and the hair, including the scalp, in particular for the protection and/or care of the skin, the lips and/or the hair, and/or for making up the skin and/or the lips.

The present invention also features the use of the subject compositions for the manufacture of products for the cosmetic treatment of the skin, the lips and the hair, including the scalp, in particular for the protection and/or care of the skin, the lips and/or the hair, and/or for making up the skin and/or the lips.

The cosmetic compositions according to the invention may for example be used as care and/or sun protection product for the face and/or the body having a liquid to semiliquid consistency, such as milks, more or less unctuous creams, gel creams and pastes. They may be optionally packaged as an aerosol and may be provided in the form of a mousse or a spray.

The compositions according to the invention in the form of vaporizable fluid lotions in accordance with the invention are applied to the skin or the hair in the form of fine particles by means of pressurized devices. The devices in accordance with the invention are well known to persons skilled in the art and comprise nonaerosol pumps or “atomizers”, the aerosol containers comprising a propellant and aerosol pumps using compressed air as propellant. The latter are described in U.S. Pat. Nos. 4,077,441 and 4,850,517.

The compositions packaged as an aerosol in accordance with the invention contain in general conventional propellants such as, for example, the hydrofluorinated compounds dichlorodifluoromethane, difluoroethane, dimethyl ether, isobutane, n-butane, propane, trichlorofluoromethane. They are preferably present in quantities ranging from 15% to 50% by weight relative to the total weight of the composition.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLES

### Example 1 and 2

### Moisturizing and Photoprotective Creams

| | Emulsion 1 | |
|---|---|---|
| | (not in | |
| | accordance | |
| | with the | Emulsion 2 |
| Ingredients | invention) | (invention) |
| Hydroxyethyldiethonium polyisobutenyl | 1.92 | 1.92 |
| triethylaminosuccinate (and) diethyl | | |
| ethanolamine (Lubrizol 5603) | | |
| Isononyl isonanoate | 2.40 | 2.40 |
| Squalane | 3.20 | 3.20 |
| Dimethicone | 0.66 | 0.66 |
| Octyl methoxycinnamate | 3 | 3 |
| 1,1-dicarboxy-(2′2′-dimethylpropyl)-4,4- | 0 | 1.32 |
| diphenylbutadiene (compound 1) | | |
| Glycerin | 5 | 5 |
| 3,3′-terephthalylidene-10,10′- | 1.32 | 0 |
| dicamphorsulphonic acid | | |
| Triethanolamine | 0.82 | 0 |
| Preservative | 1 | 1 |
| Demineralized water | qsp 100 | qsp 100 |

Emulsion 1 is formulated. 3 phases are observed.

Emulsion 2 is a homogeneous and stable cream, pleasant to apply to the skin and having good photoprotective properties.

Mode of Preparation of the Emulsions:

The aqueous phase is gradually incorporated, with stirring, into the oily phase comprising the surfactants. The emulsion is formulated at a temperature ranging from 20° C. to 60° C.

Each patent, patent application, publication and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A stable water-in-oil emulsion containing at least one polymeric surfactant comprising at least one polar part and at least one polyolefinic apolar part, and also containing at least one organic 4,4-diarylbutadiene UV-A-screening agent.

2. The water-in-oil emulsion as defined by claim 1, said polymeric surfactant comprising a polyolefinic apolar part containing at least 40 carbon atoms.

3. The water-in-oil emulsion as defined by claim 1, said polyolefinic apolar part being selected from among polymers and/or copolymers of ethylene, propylene, 1-butene, isobutene, 1-pentene, 2-methyl-1-butene, 3-methyl-1-butene, 1-hexene, 1-heptene, 1-octene, 1-decene, 1-undecene, 1-dodecene, 1-tridecene, 1-tetradecene, 1-pentadecene, 1-hexadecene, 1-heptadecene and 1-octadecene.

4. The water-in-oil emulsion as defined by claim 1, said polar part being anionic, cationic, nonionic, zwitterionic or amphoteric.

5. The water-in-oil emulsion as defined by claim 1, said polar part comprising polyalkylene glycols, polyalkylene imines, carboxylic or dicarboxylic acids, anhydrides or derivatives thereof, and mixtures thereof.

6. The water-in-oil emulsion as defined by claim 1, said polar part comprising polyoxyethylene, succinic acid or anhydride and derivatives thereof.

7. The water-in-oil emulsion as defined by claim 1, said polymeric surfactant being derived from the reaction between a polyolefin derivative and at least one carboxylic acid or anhydride selected from among maleic acid; maleic anhydride; fumaric acid; itaconic acid; citraconic acid; mesaconic acid; aconitic acid; derivatives and mixtures thereof.

8. The water-in-oil emulsion as defined by claim 1, said polymeric surfactant comprising a polyisobutylene with an optionally modified succinic endgroup.

9. The water-in-oil emulsion as defined by claim 1, said polymeric surfactant comprising the product of the reaction of maleic anhydride with polyisobutylene.

10. The water-in-oil emulsion as defined by claim 1, said polymeric surfactant reducing interfacial tension by at least 10 mN/m at a concentration of 0.01% by weight relative to the weight of the oily phase.

11. The water-in-oil emulsion as defined by claim 1, the quantity of polymeric surfactant ranging from 0.1% to 10% by weight of active material relative to the total weight of the emulsion.

12. The water-in-oil emulsion as defined by claim 1, said at least one 4,4-diarylbutadiene UV-A-screening agent having the following formula (I): 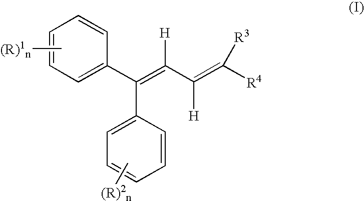
in which the diene system is of the Z,Z; Z,E; E,Z or E,E configuration or mixture of said configurations, and wherein:
R¹ and R², which may be identical or different, are each hydrogen, a C₁-C₂₀ alkyl radical, a C₂-C₁₀ alkenyl radical, a C₁-C₁₂ alkoxy radical, a C₃-C₁₀ cycloalkyl radical, a C₃-C₁₀ cycloalkenyl radical, a C₁-C₂₀ alkoxycarbonyl radical, a C₁-C₁₂ monoalkylamino radical, a C₁-C₁₂ dialkylamino radical, an aryl radical, a heteroaryl radical or a water-solubilizing substituent selected from a carboxylate residue, a sulfonate residue or an ammonium residue;
R³ is a group COOR⁵, COR⁵, CONR⁵R⁶, CN, O═S(—R⁵)═O, O═S(—OR⁵)═O, R⁷O—P—(—OR⁸)═O, a C₁-C₂₀ alkyl radical, a C₂-C₁₀ alkenyl radical, a C₃-C₁₀ cycloalkyl radical, a C₇-C₁₀ bicycloalkyl radical, a C₃-C₁₀ cycloalkenyl radical, a C₇-C₁₀ bicycloalkenyl radical, an optionally substituted C₆-C₁₈ aryl radical, an optionally substituted C₃-C₇ heteroaryl radical;
R⁴ is a group COOR⁶, COR⁶, CONR⁵R⁶, CN, O═S(—R⁶)═O, O═S(—OR⁶)═O, R⁷O—P—(—OR⁸)═O, a C₁-C₂₀ alkyl radical, a C₂-C₁₀ alkenyl radical, a C₃-C₁₀ cycloalkyl radical, a C₇-C₁₀ bicycloalkyl radical, a C₃-C₁₀ cycloalkenyl radical, a C₇-C₁₀ bicycloalkenyl radical, an optionally substituted C₆-C₁₈ aryl radical, an optionally substituted C₃-C₇ heteroaryl radical;
the radicals R⁵ to R⁸, which may be identical or different, are each hydrogen, a C₁-C₂₀ alkyl radical, a C₂-C₁₀ alkenyl radical, a C₃-C₁₀ cycloalkyl radical, a C₇-C₁₀ bicycloalkyl radical, a C₃-C₁₀ bicycloalkenyl radical, a C₇-C₁₀ cycloalkenyl radical, an optionally substituted aryl radical, an optionally substituted heteroaryl radical; and
n ranges from 1 to 3; with the proviso that the radicals R³ to R⁸ can together form, with the carbon atoms from which they depend, a C₅-C₆ ring which may be fused.

13. The water-in-oil emulsion as defined by claim 2, wherein formula (I):
n=1 or 2;
R¹ and R², which may be identical or different, are each hydrogen, a C₁-C₂₀ alkyl radical, a C₁-C₁₂ alkoxy radical, a C₁-C₁₂ monoalkylamino radical, a C₁-C₁₂ dialkylamino radical, a water-solubilizing substituent selected from among a carboxylate group, a sulfonate group or an ammonium residue;
R³ is a group COOR⁵, COR⁵, CONR⁵R⁶, a C₁-C₂₀ alkyl radical, a C₃-C₁₀ cycloalkyl radical, a C₃-C₁₀ cycloalkenyl radical, a C₇-C₁₀ bicycloalkyl radical, optionally substituted phenyl, naphthyl or thienyl;
R⁴ is a group COOR⁶, COR⁶, CONR⁵R⁶, a C₁-C₂₀ alkyl radical, a C₃-C₆ cycloalkyl radical, a C₃-C₁₀ cycloalkenyl radical, a C₇-C₁₀ bicycloalkyl radical, optionally substituted phenyl, naphthyl or thienyl; and
the radicals R⁵ and R⁶, which may be identical or different, are each hydrogen, a C₁-C₁₂ alkyl radical, a C₃-C₁₀ cycloalkyl radical, a C₃-C₁₀ cycloalkenyl radical, a C₇-C₁₀ bicycloalkyl radical, a C₃-C₁₀ bicycloalkenyl radical, optionally substituted phenyl or naphthyl.

14. The water-in-oil emulsion as defined by claim 13, wherein formula (I):
R¹ and R², which may be identical or different, are each hydrogen, a C₁-C₂₀ alkyl radical, a C₁-C₂₀ alkoxy radical, a water-solubilizing substituent selected from among a carboxylate group, a sulfonate group or an ammonium residue;
R³ is a group COOR⁵, COR⁵, CONR⁵R⁶;
R⁴ is a group COOR⁶, COR⁶, CONR⁵R⁶; and
the radicals R⁵ and R⁶, which may be identical or different, are each hydrogen, a C₁-C₁₂ alkyl radical, a C₃-C₆ cycloalkyl radical, a C₃-C₁₀ cycloalkenyl radical, a C₇-C₁₀ bicycloalkyl radical, a C₃-C₁₀ bicycloalkenyl radical, optionally substituted phenyl or naphthyl.

15. The water-in-oil emulsion as defined by claim 14, the compound of formula (I) being selected from among those of the following formula (I′): 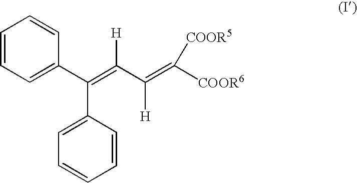
wherein the radicals R⁵ and R⁶, which may be identical or different, are each hydrogen, a C₁-C₂₀ alkyl radical, a C₃-C₆ cycloalkyl radical, a C₃-C₁₀ cycloalkenyl radical.

16. The water-in-oil emulsion as defined by claim 15, the compound of formula (I′) being 1,1-dicarboxy(2′2′dimethylpropyl)-4,4-diphenylbutadiene having the structure:

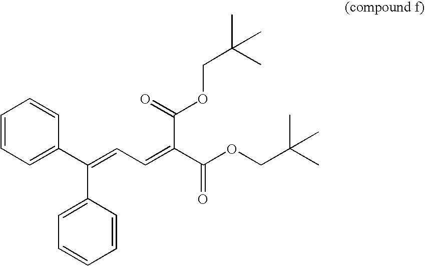

17. The water-in-oil emulsion as defined by claim 1, said at least one 4,4-diarylbutadiene UV-A-screening agent having the following formula (II): 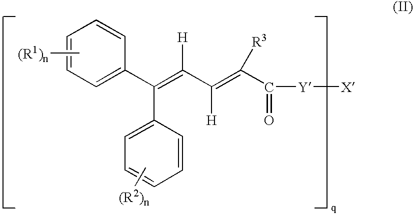
in which the diene system is of the Z,Z; Z,E; E,Z or E,E configuration or mixture of said configurations and wherein:
R¹, R², R³ and n have the meanings indicated in the formula (I);
Y′ is a group —O— or —NR⁹—;
R⁹ is hydrogen, a linear or branched C₁-C₂₀ alkyl radical, a C₂-C₁₀ alkenyl radical, a C₃-C₁₀ cycloalkyl radical, a C₇-C₁₀ bicycloalkyl radical, a C₃-C₁₀ cycloalkenyl radical, a C₇-C₁₀ bicycloalkenyl radical, an aryl radical, a heteroaryl radical;
X′ is a residue of a linear or branched, aliphatic or cycloaliphatic C₂-C₂₀ polyol comprising from 2 to 10 hydroxyl groups and having the valency q; with the proviso that the carbon chain of said residue may be interrupted by one or more sulfur or oxygen atoms, one or more imine groups, one or more C₁-C₄ alkylimino groups; and
q ranges from 2 to 10.

18. The water-in-oil emulsion as defined by claim 17, wherein said compound of formula (II):
R¹ and R², which may be identical or different, are each hydrogen, a C₁-C₁₂ alkyl radical, a C₁-C₈ alkoxy radical, a water-solubilizing substituent selected from among a carboxylate group, a sulfonate group or an ammonium residue;
R³ is a group COOR⁵, CONR⁵R⁶, CN, a C₃-C₁₀ cycloalkyl radical, a C₇-C₁₀ bicycloalkyl radical;
R⁵ and R⁶, which may be identical or different, are each a linear or branched C₁-C₂₀ alkyl radical, a C₃-C₁₀ cycloalkyl radical, a C₇-C₁₀ bicycloalkyl radical, optionally substituted naphthyl or phenyl; and
X′ is a C₂-C₂₀ polyol residue comprising from 2 to 6 hydroxyl groups.

19. The water-in-oil emulsion as defined by claim 18, wherein said compound of formula (II), X′ is an ethanol or pentaerythritol residue.

20. The water-in-oil emulsion as defined by claim 19, said compound of formula (II) being selected from among the following compounds:

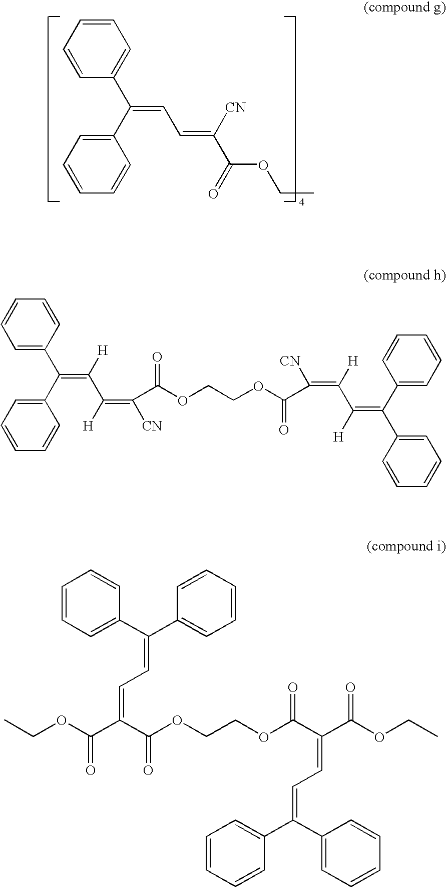

21. The water-in-oil emulsion as defined by claim 1, said at least one 4,4-diarylbutadiene compound constituting from 0.1% to 20% by weight relative to the total weight of the emulsion.

22. The water-in-oil emulsion as defined by claim 1, further comprising at least one additional organic or inorganic sunscreening agent active in the UV-A and/or UV-B regions, water-soluble, fat-soluble or insoluble in the usual cosmetic solvents.

23. The water-in-oil emulsion as defined by claim 22, comprising at least one additional organic screening agent selected from among anthranilates; cinnamic derivatives; dibenzoylmethane derivatives; salicylic derivatives, camphor derivatives; triazine derivatives; benzophenone derivatives; β,β′-diphenyl acrylate derivatives; benzotriazole derivatives; benzalmalonate derivatives; benzimidazole derivatives; imidazolines; bis-benzoazolyl derivatives; p-aminobenzoic acid (PABA) derivatives; methylenebis(hydroxyphenylbenzotriazole) derivatives; benzoxazole derivatives; screening polymers and screening silicones; dimers derived from α-alkylstyrene and mixtures thereof.

24. The water-in-oil emulsion as defined by claim 23, said at least one additional organic screening agent being selected from among:
Ethylhexyl Salicylate,
Ethylhexyl Methoxycinnamate,
Octocrylene,
Butyl Methoxydibenzoylmethane,
Phenylbenzimidazole Sulfonic Acid,
Benzophenone-3,
Benzophenone-4,
Benzophenone-5,
n-Hexyl 2-(4-diethylamino-2-hydroxybenzoyl)benzoate,
4-Methylbenzylidene camphor,
Terephthalylidene Dicamphor Sulfonic acid,
Disodium Phenyl Dibenzimidazole Tetra-sulfonate,
2,4,6-Tris(4′-diisobutyl aminobenzalmalonate)-s-triazine
Anisotriazine,
Ethylhexyl triazone,
Diethylhexyl Butamido Triazone,
Methylene bis-Benzotriazolyl Tetramethylbutylphenol,
Drometrizole Trisiloxane,
Polysilicone 15,
2,4-Bis-[5-1-(dimethylpropyl)benzoxazol-2-yl-(4-phenyl)imino]-6-(2-ethylhexyl)imino-1,3,5-triazine,
mixtures thereof.

25. The water-in-oil emulsion as defined by claim 22, comprising at least one additional inorganic screening agent selected from among metal oxide pigments or nanopigments, whether coated or uncoated.

26. The water-in-oil emulsion as defined by claim 25, said at least one additional inorganic screening agent comprising nanopigments of titanium oxide, amorphous or crystallized, in rutile and/or anatase form, iron oxide, zinc oxide, zirconium oxide or cerium oxide.

27. The water-in-oil emulsion as defined by claim 1, further comprising at least one agent for artificial bronzing and/or tanning of the skin.

28. The water-in-oil emulsion as defined by claim 1, further comprising at least one cosmetic adjuvant selected from among organic solvents, ionic or nonionic thickeners, demulcents, humectants, opacifying agents, stabilizers, emollients, silicones, insect repellents, perfumes, preservatives, surfactants, fillers, active agents, pigments, polymers, propellants, alkalinizing or acidifying agents or any other ingredient commonly employed in the cosmetic and/or dermatological field.

29. A method for the photoprotection of the skin, lips and/or hair against the damaging effects of UV-radiation, comprising topically applying thereon, a thus effective amount of a stable UV-photoprotective water-in-oil emulsion containing at least one polymeric surfactant comprising at least one polar part and at least one polyolefinic apolar part, and also containing at least one organic 4,4-diarylbutadiene UV-A-screening agent.
